(19) United States
(12) Patent Application Publication
(10) Pub. No.: US 2012/0225464 A1
(43) Pub. Date: Sep. 6, 2012

(54) Specific Arabinose Transporter of the Plant Arabidopsis Thaliana for the Construction of Pentose-Fermenting Yeasts

(76) Inventors: Eckhard BOLES, Darmstadt (DE); Thorsten SUBTIL, Rodgau (DE)

(21) Appl. No.: 13/408,442

(22) Filed: Feb. 29, 2012

(30) Foreign Application Priority Data
Mar. 4, 2011 (EP) EP 11 001 841.3

(51) Int. Cl. C12P 7/16; C12P 7/06; C12N 1/19; C12N 15/81

(52) U.S. Cl. 435/160; 435/471; 435/161; 435/254.2

(57) ABSTRACT

The present invention relates to methods for conferring upon a cell the ability to take up L-arabinose, wherein said method comprises transforming the cell with a nucleic acid molecule comprising a nucleic acid sequence that codes for a plant pentose transporter, wherein the transformation enables the cell to take up L-arabinose. The present invention further relates to yeast cells that are transformed with a nucleic acid expression construct that codes for a plant pentose transporter, wherein the expression of the nucleic acid expression construct imparts to the cells the capability to take up L-arabinose. Said cells are preferably utilized for the conversion/metabolization of biomaterial containing pentose(s), in particular arabinose, and particularly for the production of bio-based chemicals and biofuels.

### CROSS-REFERENCE TO A RELATED APPLICATION

This application claims priority to European Patent Application No. EP 11 001 841.3, filed Mar. 4, 2011, which is incorporated herein by reference in its entirety.

The present invention relates to the use of nucleic acid molecules coding for a plant pentose transporter, preferably from Arabidopsis thaliana, for the transformation of a yeast cell, wherein the transformation enables the yeast cell to specifically take up L-arabinose, and, thus for the conversion/metabolization, particularly fermentation, of biomaterial containing pentose(s), in particular arabinose, with recombinant microorganisms, and particularly for the production of bio-based chemicals and biofuels, in particular bioethanol, by means of arabinose-fermenting yeasts. The present invention further relates to yeast cells, which are transformed with a nucleic acid expression construct, which codes for a plant pentose transporter, wherein the expression of the nucleic acid expression construct imparts to the cells the capability to take up L-arabinose. Said cells are preferably utilized for the conversion/metabolization, particularly fermentation, of biomaterial containing pentose(s), in particular arabinose, and particularly for the production of bio-based chemicals and biofuels, in particular bioethanol. The present invention also relates to methods for the production of bio-based chemicals and biofuels, in particular bioethanol.

### BACKGROUND OF THE INVENTION

The beer, wine and baking yeast Saccharomyces cerevisiae has already been used for centuries for the production of bread, wine and beer owing to its characteristic of fermenting sugar to ethanol and carbon dioxide. In biotechnology, S. cerevisiae is used particularly in ethanol production for industrial purposes, in addition to the production of heterologous proteins. Ethanol is used in numerous branches of industry as an initial substrate for syntheses. Ethanol is gaining increasing importance as an alternative fuel, due to the increasingly scarce presence of oil, the rising oil prices and continuously increasing need for petrol worldwide.

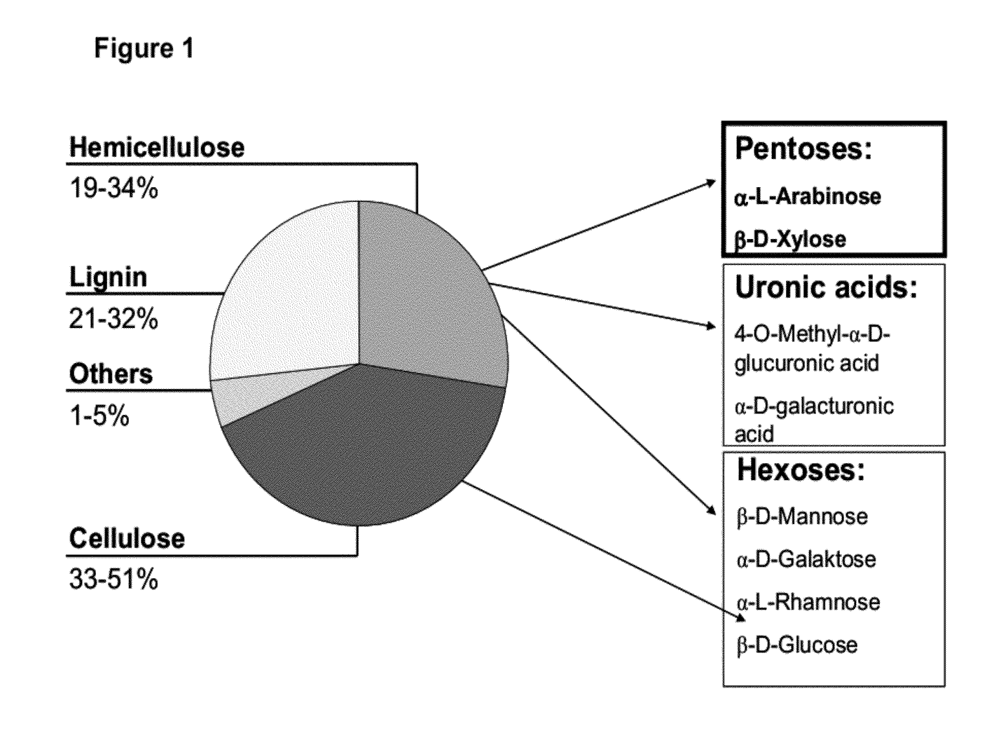

In order to make possible a favourably-priced and efficient bioethanol production, the use of biomass containing lignocellulose, such as for example straw, waste from the timber industry and agriculture and the organic component of everyday household waste, presents itself as an initial substrate. Firstly, said biomass is very convenient and secondly is present in large quantities. The three major components of lignocellulose are lignin, cellulose and hemicellulose. Hemicellulose, which is the second most frequently occurring polymer after cellulose, is a highly branched heteropolymer. It consists of pentoses (L-arabinose, D-xylose), uronic acids (4-O-methyl-D-glucuronic acid, D-galacturonic acid) and hexoses (D-mannose, D-galactose, L-rhamnose, D-glucose) (see FIG. 1). Although, hemicellulose can be hydrolized more easily than cellulose, it contains the pentoses L-arabinose and D-xylose, which can normally not be converted by the yeast S. cerevisae.

In order to be able to use pentoses for fermentations, these must firstly enter the cell through the plasma membrane. Although S. cerevisiae is not able to metabolize D-xylose, it can uptake D-xylose into the cell. However, S. cerevisiae does not have a specific transporter. The transport takes place by means of the numerous hexose transporters. The affinity of the transporters to D-xylose is, however, distinctly lower than to D-glucose (Kotter and Ciriacy, 1993). In yeasts which are able to metabolize D-xylose, such as for example P. stipitis, C. shehatae or P. tannophilus (Du Preez et al., 1986), there are both unspecific low-affinity transporters, which transport D-glucose, and also specific high-affinity proton symporters only for D-xylose (Hahn-Hagerdal et al., 2001).

In earlier experiments, some yeasts were found, such as for example Candida tropicalis, Pachysolen tannophilus, Pichia stipitis, Candida shehatae, which by nature ferment L-arabinose or can at least assimilate it. However, these yeasts lack entirely the capability of fermenting L-arabinose to ethanol, or they only have a very low ethanol yield (Dien et al., 1996). Moreover, very little is yet known about the uptake of L-arabinose. In the yeast C. shehatae one assumes a proton symport (Lucas and Uden, 1986). In S. cerevisiae, it is known from the galactose permease Gal2 that it also transports L-arabinose, which is very similar in structure to D-galactose. (Kou et al., 1970).

Alcoholic fermentation of pentoses in biotechnologically modified yeast strains of S. cerevisiae, wherein inter alia various genes of the yeast strain Pichia stipitis were used for the genetic modification of S. cerevisiae, was described in recent years particularly in connection with the fermentation of xylose. The engineering concentrated here particularly on the introduction of the genes for the initial xylose assimilation from Pichia stipitis, a xylose-fermenting yeast, into S. cerevisiae, i.e. into a yeast which is traditionally used in the ethanol production from hexose (Jin et al. 2004).

Jeppson et al. (2006) describe xylose fermentation by S. cerevisiae by means of the introduction of a xylose metabolic pathway which is either similar to that in the yeasts Pichia stipitis and Candida shehatae, which naturally use xylose, or is similar to the bacterial metabolic pathway.

Katahira et al. (2006) describe sulphuric acid hydrolysates of lignocellulose biomass such as wood chips, as an important material for the production of fuel bioethanol. In this study, a recombinant yeast strain was constructed, which is able to ferment xylose and cellooligosaccharides. For this, various genes were integrated into this yeast strain and namely for the inter-cellular expression of xylose reductase and xylitol dehydrogenase from Pichia stipitis and xylulokinase from S. cerevisiae and for the presentation of beta-glucosidase from Aspergillus acleatus on the cell surface. In the fermentation of sulphuric acid hydrolysates of wood chips, xylose and cellooligosaccharides were fully fermented by the recombinant strain after 36 hours.

Piticanen et al. (2005) describe the obtaining and characterizing of xylose chemostat isolates of a S. cervisiae strain, which over-expresses genes of Pichia stipitis coding for xylose reductase and xylitol dehydrogenase and the gene which codes endogenous xylulokinase. The isolates were obtained from aerobic chemostat cultures on xylose as the single or major carbon source. Under aerobic conditions on minimal medium with 30 g/l xylose, the growth rate of the chemostat isolates was 3 times higher than that of the original strain (0.15 h⁻¹ compared with 0.05 h⁻¹). The xylose uptake rate was increased almost two-fold. The activities of the key enzymes of the pentose phosphate metabolic pathway (transketolase, transaldolase) were increased two-fold, whilst the concentrations of their substrates (pentose-5-phosphates, sedoheptulose-7-phosphate) were lowered accordingly.

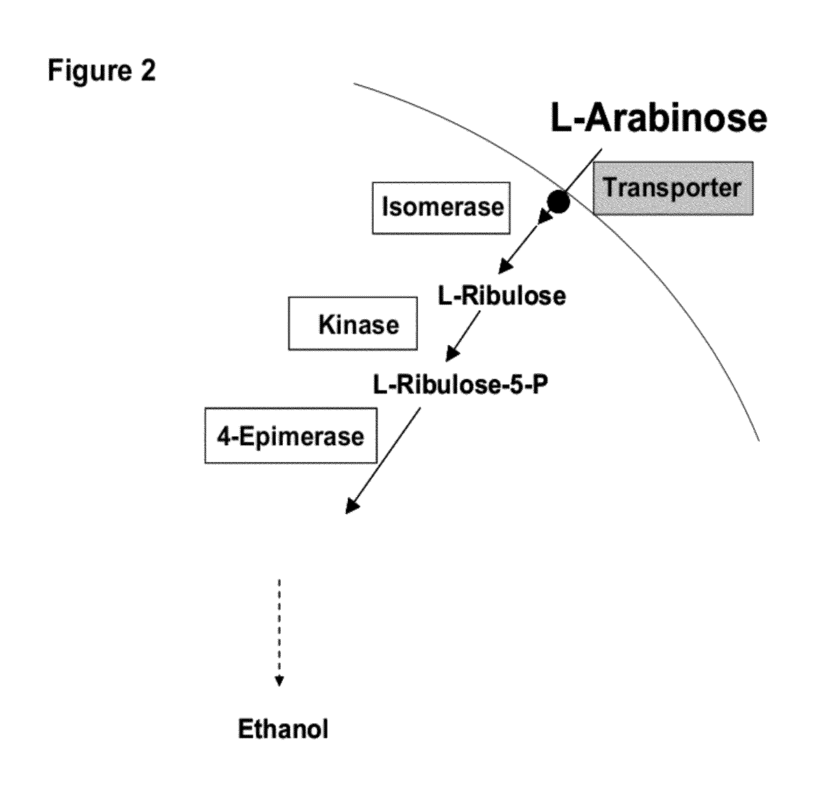

Becker and Boles (2003) describe the engineering and the selection of a laboratory strain of S. cerevisiae which is able to use L-arabinose for growth and for fermenting it to ethanol. This was possible due to the over-expression of a bacterial L-arabinose metabolic pathway, consisting of Bacillus subtilis AraA and Escherichia coli AraB and AraD and simultaneous over-expression of yeast galactose permease transporting L-arabinose in the yeast strain. Molecular analysis of the selected strain showed that the predetermining precondition for a use of L-arabinose is a lower activity of L-ribulokinase. However, inter alia, a very slow growth is reported from this yeast strain (see FIG. 2).

Wiedemann and Boles (2008) show that expressing of the codon-optimized genes of L-arabinose isomerase from Bacillus licheniformis and L-ribulokinase and L-ribulose-5-P 4-epimerase from Escherichia coli strongly improved L-arabinose conversion rates.

WO 2008/080505 discloses an arabinose transporter from Pichia stipitis, which enables yeast cells to take up L-arabinose.

There still exists a need in the art for specific pentose transporters, in particular specific L-arabinose transporters, which allow to specifically take up L-arabinose into cells, such as yeast cells, and therefore to promote the utilization and fermentation of pentoses, in particular L-arabinose.

It is thus an object of the present invention to provide improved and more specific pentose transporters, such as L-arabinose transporters.

Plant Pentose Transporter Constructs and their Use

The object is solved according to the invention by providing a nucleic acid molecule comprising a nucleic acid sequence, which codes for a plant pentose transporter, for
the transformation of a yeast cell, preferably for the recombinant expression and production of the plant pentose transporter,
the specific uptake of L-arabinose by the yeast cell, and/or
the conversion of L-arabinose by the yeast cell resulting in the formation of secondary products.

In particular for the following uses/methods:
the transformation of a yeast cell, preferably for the recombinant expression and production of the plant pentose transporter in said yeast cell,
the conversion and metabolization, particularly recombinant fermentation, of biomaterial containing pentose(s), preferably arabinose, more preferably L-arabinose,
the conversion and metabolization, particularly recombinant fermentation, of biomaterial containing hexoses and pentoses, preferably D-glucose, D-xylose and L-arabinose,
the production of bio-based chemicals or biofuels,
the production of bioalcohols, preferably bioethanol and/or biobutanol.

“Secondary products” refer to those compounds, which the cell further produces from L-arabinose after the cell has taken up the L-arabinose, such as, for example, bio-based chemicals and bioalcohols.

“Bio-based chemicals” or “biofuels” refer to chemical compounds and substances, which are obtained from biological materials and raw materials (biomass), particularly by using microorganisms.

The bio-based chemicals or biofuels can be compounds, which are selected from, but not limited to: lactic acid, acetic acid, succinic acid, malic acid, 1-butanol, isobutanol, 2-butanol, other alcohols, amino acids, 1,3-propanediol, ethylene, glycerine, a β-lactam antibiotic or a cephalosporin, alkanes, terpenes, isoprenoids or the precursor molecule amorphadiene of the antimalarial drug artemisinin.

The terms “conversion” and “metabolization” are used synonymously and refer to the metabolism of a substance or the conversion of a substance in the course of the metabolism, here: the conversion of L-arabinose by a cell, which was transformed with a nucleic acid according to the invention. A preferred conversion/metabolization is fermentation, in particular recombinant fermentation.

The nucleic acid molecules used according to the invention are recombinant nucleic acid molecules. Furthermore, nucleic acid molecules used according to the invention comprise dsDNA, ssDNA, PNA, CNA, RNA or mRNA or combinations thereof.

The plant pentose transporter according to the invention originates from the plant Arahidopsis, preferably Arahidopsis thaliana.

The plant pentose transporter is preferably the transporter Stp2 from Arahidopsis thaliana. Stp2 is a protein of 498 amino acids (see SEQ ID NO. 1).

In this invention, a specific L-arabinose transporter gene from A. thaliana was identified by using a test system (see examples). Truernit et al., 1999 identified the transporter Stp2 from Arahidopsis thaliana as a proton symporter with a high affinity for galactose. The inventors designed and used the plasmid pTHStp2, which has the ORF from AtSTP2 localized on it. Yeast cells of the strain MKY06 were transformed with the plasmid pTHStp2, which is responsible for a specific growth on L-arabinose but not on D-glucose. The possibility that the obtained growth of the transformed yeast cells was brought about by a genomic mutation in MKY06 was ruled out. After a selection on the loss of the plasmid pTHStp2 no further growth was established with renewed smearing on L-arabinose medium.

Thus, the plant pentose transporter used according to the invention allows the specific in vitro and/or in vivo transport and uptake of L-arabinose into the transformed yeast cell, however, it does not transport D-glucose. This allows a respective transformed yeast cell or strain to convert and metabolize, particularly recombinantly ferment, biomaterial which contains pentose(s) (preferably L-arabinose), but also biomaterial which contains hexoses and pentoses, preferably D-glucose, D-xylose and L-arabinose. Wherein the respective transformed yeast cell or strain is imparted the capability to take up L-arabinose in the presence of hexoses, particularly D-glucose.

Due to the specificity of the plant pentose transporter used according to the invention, after expression in existing ethanol-producing systems, such as respective recombinant yeast cells/strains, the uptake rate for L-arabinose can be improved, because firstly with high L-arabinose concentrations the competitive situation with respect to glucose is improved, and secondly with low L-arabinose concentrations the transport of L-arabinose becomes more efficient owing to the high affinity.

The plant pentose transporter according to the invention preferably comprises an amino acid sequence, which is at least 70% identical, preferably at least 80% identical, more preferably at least 90% identical, even more preferably at least 95% identical and yet more preferably 99% identical to the amino acid sequence of SEQ ID NO: 1 and has an in vitro and/or in vivo pentose transport function (in particular an in vitro and/or in vivo L-arabinose transport function) or is identical to the amino acid sequence of SEQ ID NO: 1.

As used herein, the term “percent (%) identical” refers to sequence identity between two amino acid sequences. Identity can be determined by comparing a position in both sequences, which may be aligned for the purpose of comparison. When an equivalent position in the compared sequences is occupied by the same amino acid, the molecules are considered to be identical at that position.

The plant pentose transporter used according to the invention also comprises amino acid sequences that are functional equivalents of the amino acid sequence of SEQ ID NO: 1 having an in vitro and/or in vivo pentose transport function, in particular an in vitro and/or in vivo L-arabinose transport function.

As used herein, the term “functional equivalent” refers to amino acid sequences that are not 100% identical to the amino acid sequence of SEQ ID NO. 1 and comprise amino acid additions and/or insertions and/or deletions and/or substitutions and/or exchanges, which do not alter or change the activity or function of the protein as compared to the protein having the amino acid sequence of SEQ ID NO: 1, i.e. an “functional equivalent”, for example, encompasses an amino acid sequence with conservative amino acid substitutions or smaller deletions and/or insertions as long as these modifications do not substantially affect the in vitro and/or in vivo L-arabinose transport function.

Generally, a person skilled in the art is aware of the fact that some amino acid exchanges in the amino acid sequence of a protein do not have an influence on the (secondary or tertiary) structure, function and/or activity of that protein. Amino acid sequences with such “neutral” amino acid exchanges as compared to the amino acid sequences disclosed herein fall within the scope of the present invention.

When the nucleic acid sequence coding for the plant pentose transporter is expressed in a yeast cell, the yeast cell is imparted the capability to take up L-arabinose, which then may be metabolized further. Through this, the cell is able to grow on L-arabinose as a carbon source. The present invention provides methods for conferring upon a cell the ability to take up L-arabinose, wherein said method comprises transforming the cell with a nucleic acid molecule comprising a nucleic acid sequence, which codes for a plant pentose transporter, wherein the transformation enables the cell to take up L-arabinose, wherein the cell is a yeast cell, and wherein the plant pentose transporter originates from Arabidopsis and comprises an amino acid sequence-that is at least 70%, preferably at least 80%, more preferably at least 90% identical to the amino acid sequence of SEQ ID NO: 1 and has an in vitro and/or an in vivo pentose transport function, or a functional equivalent of SEQ ID NO: 1 having an in vitro and/or an in vivo pentose transport function.

The nucleic acid sequence coding for the plant pentose transporter preferably comprises a nucleic acid sequence, which is at least 70% identical, preferably at least 80% identical, more preferably at least 90% identical, even more preferably at least 95% identical and yet more preferably 99% identical or identical to the nucleic acid sequence of SEQ ID NO: 2.

The nucleic acid molecules according to the invention preferably comprise nucleic acid sequences, which are identical with the naturally occurring nucleic acid sequence or are codon-optimized for the use in a host cell.

The nucleic acid molecule used according to the present invention is preferably a nucleic acid expression construct.

Nucleic acid expression constructs according to the invention are expression cassettes comprising a nucleic acid molecule according to the invention, or expression vectors comprising a nucleic acid molecule according to the invention or an expression cassette, for example.

A nucleic acid expression construct preferably comprises regulatory sequences, such as promoter and terminator sequences, which are operatively linked with the nucleic acid sequence coding for the plant pentose transporter.

Preferred promoter sequences are HXT7, truncated HXT7, PFK1, FBA1, PGK1, ADH1 and TDH3. Preferred terminator sequences are CYC1, FBA1, PGK1, PFK1, ADH1 and TDH3. The nucleic acid expression construct may further comprise 5′ and/or 3′ recognition sequences and/or selection markers.

A preferred selection marker is a LEU2 marker gene, a URA3 marker gene, a TRP1 marker gene, a HIS3 marker gene and a dominant antibiotic-resistance marker gene. A preferred dominant antibiotic-resistance marker gene is a gene, which imparts resistances to geneticin, hygromycin and nourseothricin.

The yeast cell is preferably a member of a genus selected from the group of Saccharomyces, Kluyveromyces, Candida, Pichia, Schizosaccharomyces, Hansenula, Kloeckera, Schwanniomyces, Arxula and Yarrowia.

The yeast cell is more preferably a member of a species selected from the group of S. cerevisiae, S. bulderi, S. barnetti, S. exiguus, S. uvarum, S. diastaticus, K. lactis, K. marxianus, K. fragilis, H. polymorpha, P. pastoris and Y. lipolytica, such as S. cerevisiae, K. lactis, H. polymorpha, P. pastoris or Y. lipolytica.

In a preferred embodiment, the yeast cell further contains nucleic acid molecules which code for proteins of an arabinose metabolic pathway, in particular for L-ribulokinase, L-ribulose-5-P 4-epimerase. L-arabinose-isomerase.

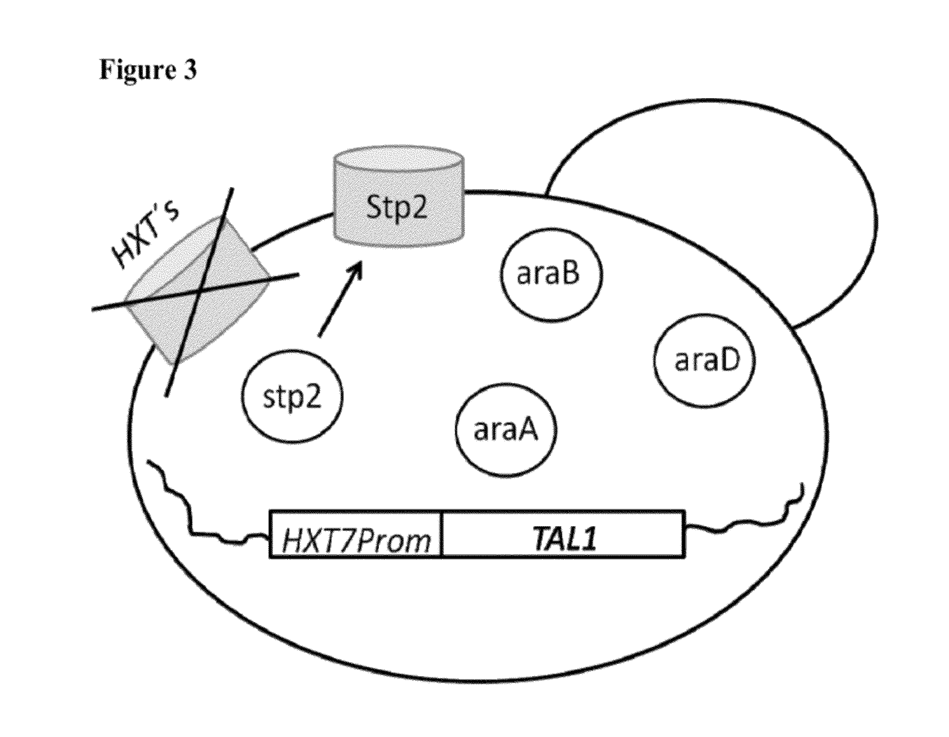

Preferred are proteins of the bacterial arabinose metabolic pathway, in particular E. coli araB L-ribulokinase, E. coli araD L-ribulose-5-P 4-epimerase and B. licheniformis araA L-arabinose-isomerase. See also FIGS. 2 and 3.

Depending on the intended use of the yeast cell, said yeast cell can contain, express or overexpress further nucleic acid sequences coding for further proteins, such as transaldolase TAL1 and/or TAL2, transketolase TKL1 and/or TKL2, D-ribulose-5-phosphate 3-epimerase RPE1, ribose-5-phosphate ketol-isomerase RKI1 or the corresponding sequences from other organisms encoding the same enzyme activities.

Preferably, a yeast cell according to this invention is modified by the introduction and expression or the genes araA (L-arabinose-isomerase), araB (L-ribulokinase) and araD (L-ribulose-5-P-4-epimerase) and in addition over-expresses a TAL1 (transaldolase) gene as described for example by the inventors in EP 1 499 708 B1, and in addition to this contains at least one nucleic acid molecule according to the invention.

Preferably, a method according to the invention is used for
the conversion and metabolization, particularly recombinant fermentation, of biomaterial containing pentose(s), preferably arabinose, more preferably L-arabinose,
the conversion and metabolization, particularly recombinant fermentation, of biomaterial containing hexoses and pentoses, preferably D-glucose, D-xylose and L-arabinose,
the production of bio-based chemicals or biofuels, and/or
the production of bioalcohols, preferably bioethanol and/or biobutanol.

### Yeast Cells Specifically Taking Up L-Arabinose

The object is solved according to the invention by providing yeast cells, which are transformed with a nucleic acid expression construct coding for a plant pentose transporter.

A yeast cell according to the invention is transformed with a nucleic acid expression construct comprising:

(a) a nucleic acid sequence coding for a plant pentose transporter,
(b) regulatory elements operatively linked with the nucleic acid sequence, allowing for the expression of the plant pentose transporter in the yeast cell.

Thereby, the expression of the nucleic acid expression construct imparts to the yeast cell the capability to take up L-arabinose.

Preferably, the expression of the nucleic acid expression construct imparts to the yeast cell the capability to take up L-arabinose in the presence of hexoses, particularly D-glucose. Preferably, the expression of the nucleic acid expression construct imparts to the yeast cell the capability to take up L-arabinose but not D-glucose.

As disclosed above, the plant pentose transporter used according to the invention allows the specific in vitro and/or in vivo transport and uptake of L-arabinose into a transformed yeast cell, however, it does not transport D-glucose. This allows the respective transformed yeast cell or strain to convert and metabolize, particularly recombinantly ferment, biomaterial which contains pentose(s) (preferably L-arabinose), but also biomaterial which contains hexoses and pentoses, preferably D-glucose, D-xylose and L-arabinose. Wherein the respective transformed yeast cell or strain is imparted the capability to take up L-arabinose in the presence of hexoses, particularly D-glucose.

Due to the specificity of the plant pentose transporter used according to the invention, after expression in existing ethanol-producing systems, such as respective recombinant yeast cells/strains, the uptake rate for L-arabinose can be improved, because firstly with high L-arabinose concentrations the competitive situation with respect to glucose is improved, and secondly with low L-arabinose concentrations the transport of L-arabinose becomes more efficient owing to the high affinity.

The plant pentose transporter according to the invention originates from Arabidopsis, preferably Arabidopsis thaliana. The plant pentose transporter according to the invention comprises an amino acid sequence, which is at least 70% identical, preferably at least 80% identical, more preferably at least 90% identical, even more preferably at least 95% identical and yet more preferably 99% identical and has an in vitro and/or in vivo pentose transport function (in particular an in vitro and/or in vivo L-arabinose transport function), as disclosed and defined herein, or is identical to the amino acid sequence of SEQ ID NO: 1, as disclosed and defined herein. The plant pentose transporter used according to the invention also comprises amino acid sequences that are functional equivalents of the amino acid sequence of SEQ ID NO: 1 having an in vitro and/or in vivo pentose transport function, in particular an in vitro and/or in vivo L-arabinose transport function, as disclosed and defined herein.

The regulatory elements, such as promoter and terminator sequences, are operatively linked with the nucleic acid sequence coding for the plant pentose transporter and allow for the expression or the plant pentose transporter in the yeast cell.

Preferred promoter sequences are HXT7, truncated HXT7, PFK1, FBA1, PGK1, ADH1 and TDH3. Preferred terminator sequences are CYC1, FBA1, PGK1, PFK1, ADH1 and TDH3. The nucleic acid expression construct may further comprise 5′ and/or 3′ recognition sequences and/or selection markers. A preferred selection marker is a LEU2 marker gene, a URA3 marker gene, a TRP1 marker gene, a HISS marker gene and a dominant antibiotic-resistance marker gene. A preferred dominant antibiotic-resistance marker gene is a gene, which imparts resistances to geneticin, hygromycin and nourseothricin.

In a preferred embodiment, the nucleic acid expression construct with which a yeast cell according to the invention is transformed is a nucleic acid molecule according to the invention, as defined herein and above.

The yeast cell is preferably a member of a genus selected from the group of Saccharomyces, Kluyveromyces, Candida, Pichia, Schizosaccharomyces, Hansenula, Kloeckera, Schwanniomyces, Arxula and Yarrowia.

The yeast cell is more preferably a member of a species selected from the group of S. cerevisiae, S. bulderi, S. barnetti, S. exiguus, S. uvarum, S. diastaticus, K. lactis, K. marxianus, K. fragilis, H. polymorpha, P. pastoris and Y. lipolytica, such as S. cerevisiae, K. lactis, H. polymorpha, P. pastoris or Y. lipolytica.

In a preferred embodiment, the yeast cell further contains nucleic acid molecules which code for proteins of an arabinose metabolic pathway, in particular for L-ribulokinase, L-ribulose-5-P 4-epimerase, L-arabinose-isomerase.

Preferred are proteins of the bacterial arabinose metabolic pathway, in particular E. coli araB L-ribulokinase, E. coli araD L-ribulose-5-P 4-epimerase and B. licheniformis araA L-arabinose-isomerase. See also FIGS. 2 and 3.

Depending on the intended use of the yeast cell, said yeast cell can contain, express or overexpress further nucleic acid sequences coding for further proteins, such as transaldolase TAL1 and/or TAL2, transketolase TKL1 and/or TKL2, D-ribulose-5-phosphate 3-epimerase RPE1, Ribose-5-phosphate ketol-isomerase RKI1 or the corresponding sequences from other organisms encoding the same enzyme activities.

Preferably, a yeast cell according to this invention is modified by the introduction and expression of the genes araA (L-arabinose-isomerase), araB (L-ribulokinase) and araD (L-ribulose-5-P-4-epimerase) and in addition over-expresses a TAL1 (transaldolase) gene, as described for example by the inventors in EP 1 499 708 B1, and in addition to this contains at least one nucleic acid molecule according to the invention.

A yeast cell according to the invention is more preferably the strain Ethanol Red™ or Lallemand1 or other yeast strains commonly used in the bioethanol industry.

The yeast cell according to the invention is preferably a cell maintained in a cell culture or a cultured cell.

The yeast cells according to the invention are transiently or stably transformed with the nucleic acid expression construct or the nucleic acid molecule, as defined herein.

In one embodiment, a yeast cell according to the invention furthermore expresses one or more enzymes, which impart to the cell the capability to produce one or more further metabolization products.

A “further metabolization product” is preferably selected from, but not limited to, the group of bio-based chemicals or biofuels, such as lactic acid, acetic acid, succinic acid, malic acid, 1-butanol, isobutanol, 2-butanol, other alcohols, amino acids, 1,3-propanediol, ethylene, glycerol, a β-lactam antibiotic or a cephalosporin, alkanes, terpenes, isoprenoids or the precursor molecule amorphadiene of the antimalarial drug artemisinin.

The object is solved according to the invention by using the yeast cells according to the invention for
the conversion and metabolization, particularly recombinant fermentation, of biomaterial containing pentose(s), preferably arabinose, more preferably L-arabinose,
the conversion and metabolization, particularly recombinant fermentation, of biomaterial containing hexoses and pentoses, preferably D-glucose, D-xylose and L-arabinose,
the production of bio-based chemicals or biofuels,
the production of bioalcohols, preferably bioethanol and/or biobutanol.

Preferably, the present invention provides a method for
the conversion and metabolization, particularly recombinant fermentation, of biomaterial containing pentose(s), preferably arabinose, more preferably L-arabinose,
the conversion and metabolization, particularly recombinant fermentation, of biomaterial containing hexoses and pentoses, preferably D-glucose, D-xylose and L-arabinose,
the production of bio-based chemicals or biofuels,
the production of bioalcohols, preferably bioethanol and/or biobutanol.
   wherein said method comprises the use of a yeast cell according to the invention.

### Methods for the Production of Biomolecules

The object is furthermore solved according to the invention by providing a method for the production of bioalcohol(s).

The method according to the invention comprises the following steps:

(a) contacting a medium containing a pentose source with a cell according to the invention, which converts pentose to a bioalcohol,
(b) optionally obtaining the bioalcohol,

The pentose is preferably arabinose, more preferably L-arabinose.

The bioalcohol is preferably bioethanol and/or biobutanol. The bioalcohol is obtained by isolation, for example.

The medium may also contain a further carbon source, particularly hexose, more particularly glucose.

The object is furthermore solved according to the invention by providing a method for the production of metabolization product(s)/bio-based chemicals or biofuels.

The method according to the invention comprises the following steps:

(a) contacting a medium containing a pentose source with a cell according to claim 13, which converts pentose to produce the metabolization product,
(b) optionally obtaining the metabolization product,

The pentose is preferably arabinose, more preferably L-arabinose.

The metabolization product is preferably selected from the group of bio-based chemicals or biofuels, such as lactic acid, acetic acid, succinic acid, malic acid, 1-butanol, isobutanol, 2-butanol, other alcohols, amino acids, 1,3-propanediol, ethylene, glycerol, a β-lactam antibiotic or a cephalosporin, alkanes, terpenes, isoprenoids or the precursor molecule amorphadiene of the antimalarial drug artemisinin.

The metabolization product is obtained by isolation, for example.

The medium may also contain a further carbon source, particularly hexose, more particularly glucose.

The inventors have identified by a gene screening a novel specific L-arabinose transporter, the nucleotide and protein sequence of which is presented herein (see SEQ ID NOs: 1 and 2). For this, reference is also to be made to the examples and figures.

Due to the specificity of this novel transporter, after expression in existing ethanol-producing systems the uptake rate for L-arabinose can be improved, because firstly with high L-arabinose concentrations the competitive situation with respect to glucose is improved, and secondly with low L-arabinose concentrations the transport of L-arabinose becomes more efficient owing to a high affinity.

### Uptake of L-arabinose

So that the pentose L-arabinose can be metabolized by S. cerevisiae, it must firstly be taken up by the cell. Only little is known with regard to this uptake. Hitherto, no genes are known in eukaryontes, which code for specific L-arabinose transporters. All hexose transporters tested for the pentose D-xylose have a much higher affinity to hexoses than to D-xylose. For L-arabinose, a similar situation is assumed. Of all strains constructed hitherto, which can utilize pentoses (D-xylose or L-arabinose), a relatively slow growth is reported. Above all, the slow and poor uptake of the pentoses is named as a reason for this (Becker and Boles, 2003; Richard et al. 2002). In fermentations in a sugar mixture, consisting of D-glucose and D-xylose or D-glucose and L-arabinose, the sugars are not converted simultaneously. Due to the high affinity of the transporters for D-glucose, D-glucose is metabolized at first. A so-called Diauxic shift occurs. Only after the D-glucose is exhausted is the pentose converted in a second, distinctly slower growth phase (Kuyper et al., 2005a; Kuyper et al., 2005b). The absence of specific transporters for pentoses is given as an explanation.

Novel Specific L-Arabinose Transporter from A. Thaliana

For industrial applications, it would be ideal if the microorganism which was used could convert all the sugars present in the medium as far as possible simultaneously (Zaldivar et al., 2001). In order to achieve this, specific transporters for each sugar type would be of great benefit. None were known hitherto particularly for L-arabinose.

In this invention, it has been successful with a test system (see examples) to find a specific L-arabinose transporter gene from A. thaliana. Truernit et al., 1999 identified the transporter Stp2 from Arabidopsis thaliana as a proton symporter with a high affinity for galactose. Truernit et al. furthermore characterized Stp2 in the yeast Schizosaccharomyces pombe, where the following sugars were transported: D-glucose, D-galactose, D-xylose, D-mannose, 3-O-methylglucose and D-fructose. The inventors used the plasmid pTHStp2, containing the ORF from AtSTP2, and found it to be responsible for a specific growth of respective transformed S. cerevisiae (strain MKY06) on L-arabinose but not on D-glucose, and in a comparable growth-based screen with overexpression of a xylose isomerase also not on xylose. The possibility that the obtained growth was brought about by a genomic mutation in MKY06 was ruled out. After a selection on the loss of the plasmid pTHStp2 no further growth was established with renewed smearing on L-arabinose medium. The results demonstrate that AtStp2 is able to confer to the yeast cells the property to take up arabinose with a higher specificity and with a higher affinity than normal yeast cells do. This property is important for the fermentation in particular of mixtures of hexose and pentose sugars, in particular D-glucose, D-xylose and L-arabinose, and in particular of plant biomass hydrolysates where normally D-glucose is present in much higher concentrations than L-arabinose. The specific properties of AtStp2 in the yeast cells will allow the fermentation of L-arabinose in the presence of D-glucose, and even at low L-arabinose concentrations.

Recently, Verho et al., 2011 describe the cloning of two genes LAT1 and LAT2 of the L-arabinose fermenting yeast Ambrosiozyma monospora. In the present inventors' test system, these genes, however, did not proof to be coding for arabinose transporters.

Furthermore, a multitude of experimental obstacles and difficulties were to be overcome in the locating and provision of the transporter according to the invention, which can also be seen in greater detail from the examples and figures.
In the initial strain EBY.VW4000, a total of 21 monosaccharide transporter genes had to be deleted.
Furthermore, in this strain TAL1 had to be over-expressed genomically.
The establishing of the optimum growth conditions for carrying out the screen proved to be very difficult and time-consuming.
The transporter according to the invention is the second described specific arabinose transporter of cukaryots. It has a higher specificity for arabinose than the transporter AraT of P. stipitis (as disclosed in WO 2008/080505) because it transports no glucose in the strain MKY06-3P in contrast to AraT which transports small amounts of glucose when overexpressed,
The concern is with a heterologously expressed transporter which is at the same time functionally incorporated in the plasma membrane of S. cerevisiae, which is not implicitly to be expected.

Some reports exist with regard to the difficulties concerning heterologously expressed transporters, see on this subject Chapter 2 in the book “Transmembrane Transporters” (Boles, 2002) and the article by Wieczorke et al., 2003.

Further biomass with significant amounts of arabinose (source of the data: U.S. Department of Energy http://www.eere.energy.gov/biomass/progs/search1.cgi):

| Type of biomass | L-arabinose [%] |
|---|---|
| Switchgrass | 3.66 |
| Large bothriochloa | 3.55 |
| Tall fescue | 3.19 |
| Robinia | 3 |
| Corn stover | 2.69 |
| Wheat straw | 2.35 |
| Sugar can bagasse | 2.06 |
| Chinese lespedeza | 1.75 |
| Sorghum bicolor | 1.65 |

The arabinose transporter according to the invention is also of great importance for its utilization.

Possibilities for use of a functional and at the same time specific arabinose transporter in the yeast S. cerevisiae are firstly the production of bioethanol and the production of high-grade precursor products for further chemical syntheses.

The following list originates from the study “Top Value Added Chemicals From Biomass” (see www1.eere.energy.gov/biomass/pdfs/35523.pdf). Here, 30 chemicals were categorized as being particularly valuable, which can be produced from biomass.

| Number of C | |
|---|---|
| atoms | Top 30 Candidates |
| 1 | hydrogen, carbon monoxide |
| 2 | |
| 3 | glycerol, 3-hydroxypropionic acid, lactic acid, |
| | malonic acid, propionic acid, serine |
| 4 | acetoin, asparaginic acid, fumaric acid, |
| | 3-hydroxybutyrolactone, malic acid, succinic acid, threonin |
| 5 | arabitol, furfural, glutamic acid, itaconic acid, |
| | levulinic acid, proline, xylitol, xylonic acid |
| 6 | aconitic acid, citrate, 2,5-furandicarboxylic acid, |
| | glucaric acid, lysine, levoglucosan, sorbitol |

As soon as these chemicals are produced from lignocelluloses by bioconversion (e.g. fermentations with yeasts), it is important to have a specific transporter for the hemicellulose arabinose.

The present invention is further clarified in the following figures, sequences and examples, without however being restricted thereto. The cited references are fully included herewith by reference. In the sequences and figures there are shown:

SEQ ID NO: 1 the protein sequence of the Stp2,
SEQ ID NO: 2 the nucleotide sequence of the open reading frame (ORF) of Stp2.

FIG. 1. Composition of the biomass

The second most frequently occurring hemicellulose is a highly branched polymer consisting of pentoses, uronic acids and hexoses. The hemicellulose consists in a large proportion of the pentoses xylose and arabinose.

FIG. 2. Scheme for the use of L-arabinose in recombinant S. cerevisiae by integration of a bacterial L-arabinose metabolic pathway.

FIG. 3. Construction of the yeast strain MKY06-3P+pTHStp2 according to the invention.

The initial strain for the construction of MKY06-3P was the yeast strain EBY.VW4000, in which all hexose transporter genes (HXTs) were deleted. In this strain, the endogenous transaldolase TAL1 was over-expressed by the exchange of the native promoter for the shortened HXT7 promoter (HXT7-Prom). This led to the strain MKY06. Into this strain, the plasmids p4231H7-synthIso, p424H7-synthKin and p425H7-synthEpi were transformed for the arabinose metabolism (=MKY06-3P). In addition, the plasmid pTHStp2 (AtStp2), which codes for the arabinose transporter according to the invention from Arabidopsis thaliana was also transformed into this strain and, thus, the strain MKY06-3P+pTHStp2 was obtained. The transporter is expressed and is functionally incorporated into the plasma membrane.

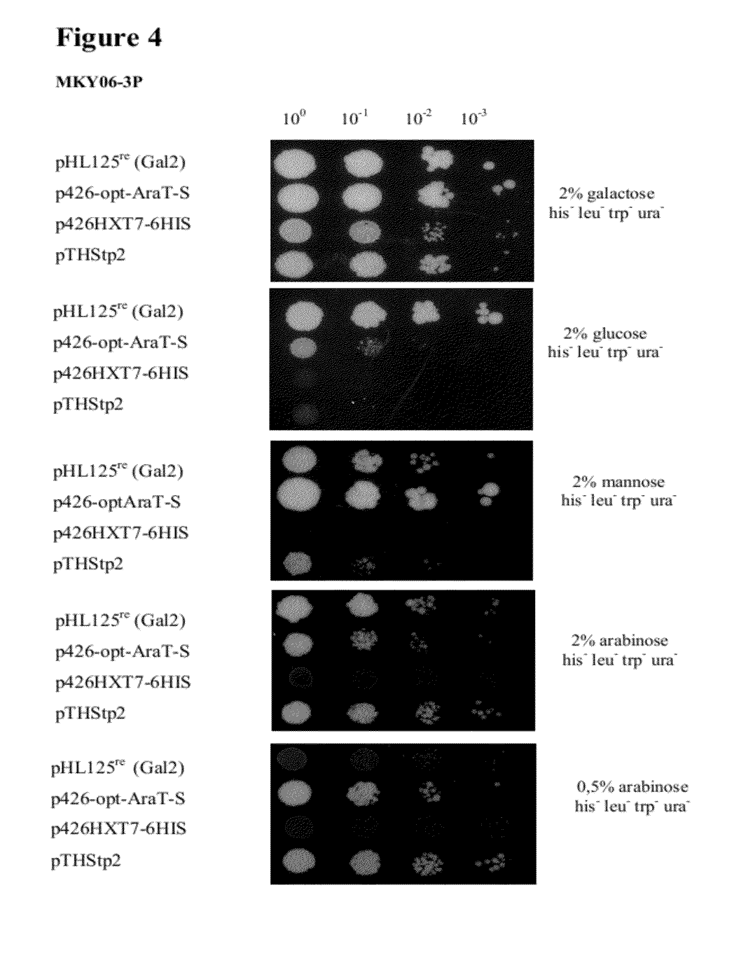

FIG. 4. Dropping of the MKY06-3P with the plasmids for the L-arabinose metabolism and the found L-arabinose transporters on various carbon sources.

As a negative control (−), instead of the pTHStp2 plasmid, the p426-HXT7-6HIS was transformed in as a negative control and as positive controls (+) the pHL125^{re} and p426-optAraT-S.

1: pHL125″ 2:p426-opt-AraT-S 3:p426HXT7-6HIS 4:pTHStp2

Medium with 2% D-galactose, 2% D-glucose, 2% mannose, 2% L-arabinose, 0.5% L-arabinose

All SC medium plates were incubated at 30° C. The L-arabinose plates show growth after 10 days and all other plates after 3 days.

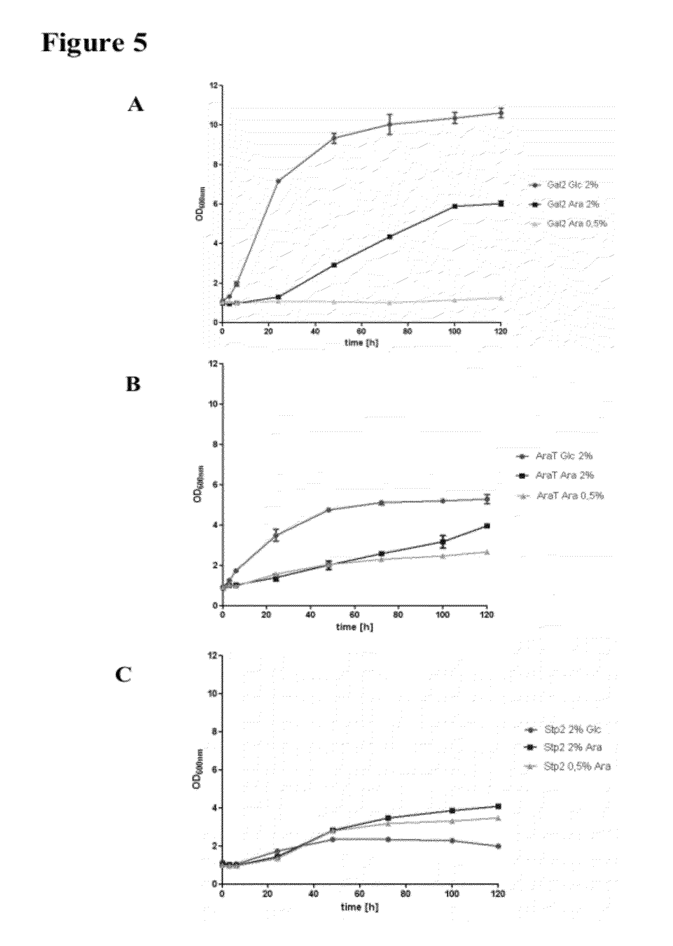

FIG. 5. Growth behaviour on glucose and arabinose with the use of the arabinose transporter.

Growth of the MKY06-3P, which additionally also contains the plasmids pHL125^{re} (A), p426-opt-AraT-S (B) and pTHSTp2 (C) in SC medium with 2% glucose, 2% or 0.5% L-arabinose under aerobic conditions. The strains with the various plasmids were adducted in medium with the respective carbon source and inoculated with an OD₆₀₀ₙₘ=1 in 50 ml SC medium. The incubation took place in 300 ml shaking flasks under aerobic conditions at 30° C. Samples were taken several times during the 120 hours to determine the optical density.

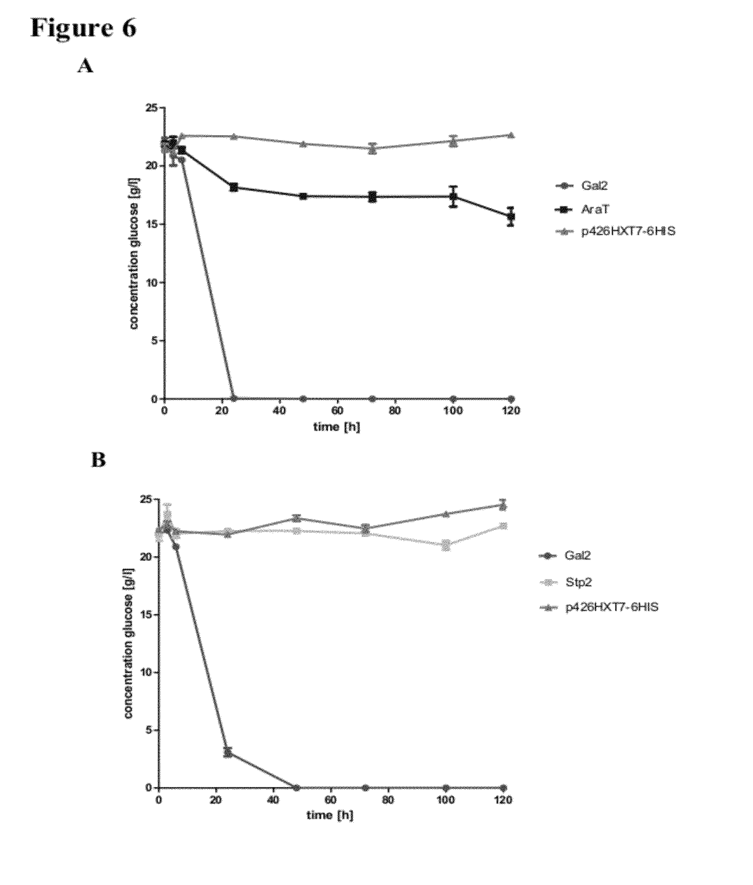

FIG. 6. Comparison of glucose fermentation of AraT and Stp2 on 2% glucose media.

Fermentation analysis of the MKY06-3P containing the plasmids p426-opt-AraT-S (A) or pTHSTp2 (B) in SC medium with 2% glucose under aerobic conditions. As controls the strain MKY06-3P was used containing the plasmids pHL125^{re} (positive) or p426HXT7-6HIS (negative). The strains with the various plasmids were adducted in SC medium with 2% glucose and inoculated with an OD₆₀₀ₙₘ=1 in 50 ml SC medium. The incubation took place in 300 ml shaking flasks under aerobic conditions at 30° C. Samples were taken several times during the 120 hours to determine the sugar concentration via HPLC analysis.

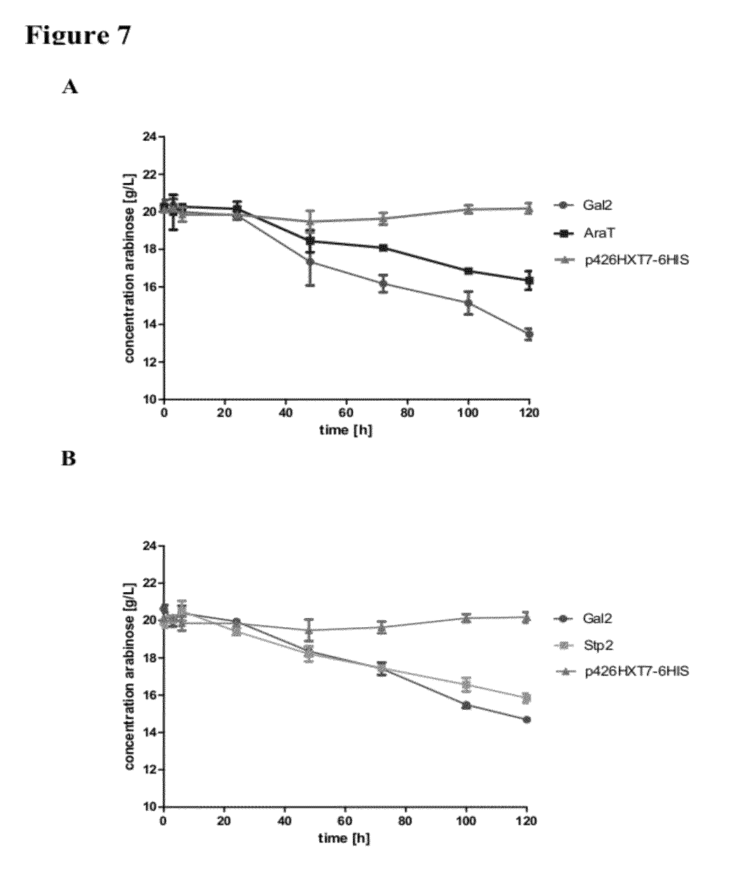

FIG. 7. Comparison of arabinose fermentation of AraT and Stp2 on 2% arabinose media.

Fermentation analysis of the MKY06-3P containing the plasmids p426-opt-AraT-S (A) or pTHSTp2 (B) in SC medium with 2% arabinose under aerobic conditions. As controls the strain MKY06-3P was used containing the plasmids pHL125^{re} (positive) or p426HXT7-6HIS (negative). The strains with the various plasmids were adducted in SC medium with 2% arabinose and inoculated with an OD₆₀₀ₙₘ=1 in 50 ml SC medium. The incubation took place in 300 ml shaking flasks under aerobic conditions at 30° C. Samples were taken several times during the 120 hours to determine the sugar concentration via HPLC analysis.

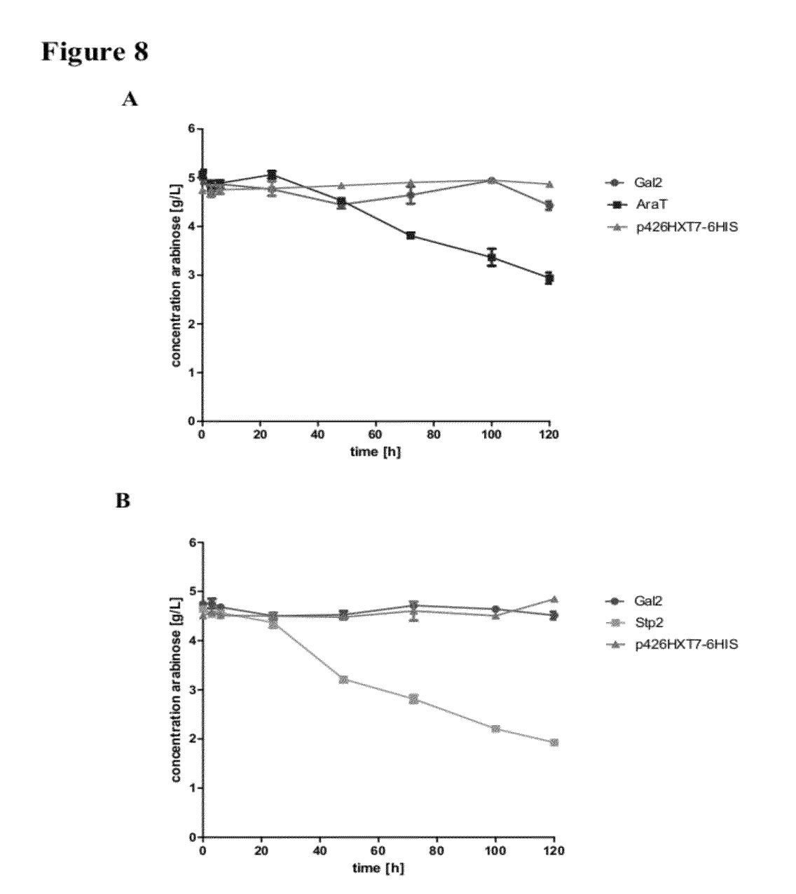

FIG. 8. Comparison of arabinose fermentation of AraT and Stp2 on 0.5% arabinose media.

Fermentation analysis of the MKY06-3P containing the plasmids p426-opt-AraT-S (A) or pTHSTp2 (B) in SC medium with 0.5% arabinose under aerobic conditions. As controls the strain MKY06-3P was used containing the plasmids pHL125^{re} (positive) or p426HXT7-6HIS (negative). The strains with the various plasmids were adducted in SC medium with 0.5% arabinose and inoculated with an OD₆₀₀ₙₘ=1 in 50 ml SC medium. The incubation took place in 300 ml shaking flasks under aerobic conditions at 30° C. Samples were taken several times during the 120 hours to determine the sugar concentration via HPLC analysis.

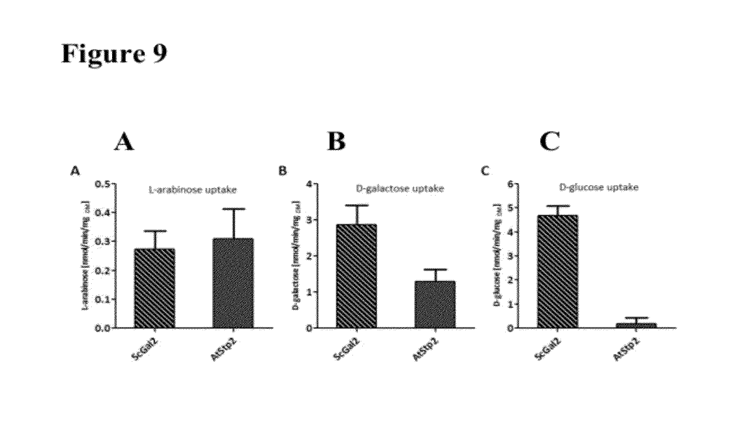

FIG. 9. Comparison of initial sugar uptake of strain MKY06 expressing Stp2 and Gal2

Initial rates of sugar uptake of strain MKY06 expressing Stp2 (AtStp2) or Gal2 (ScGal2). Cells were grown on D-galactose or maltose (empty vector), harvested and incubated with the radioactively labeled sugars: 10 mM L-arabinose (A), 10 mM D-galactose (B) and 10 mM D-glucose (C) for 2 minutes (L-arabinose) or 1 minute (D-galactose, D-glucose). The background values determined with cells containing the empty vector were subtracted.

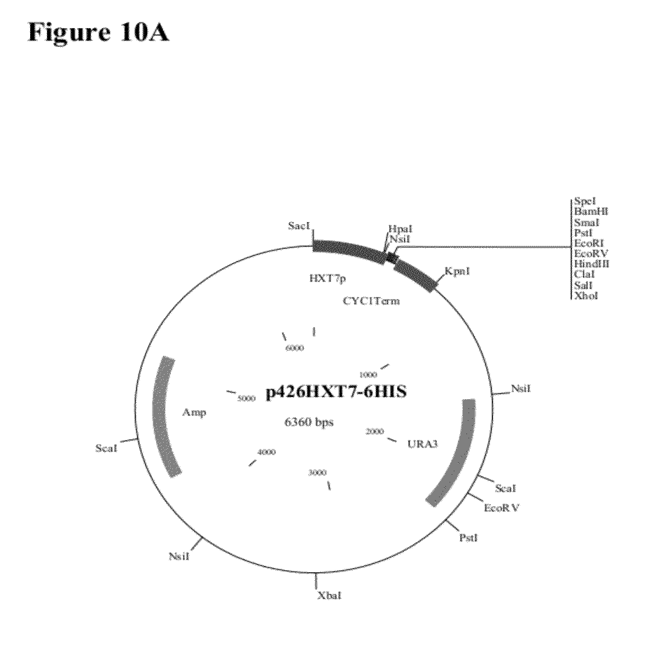

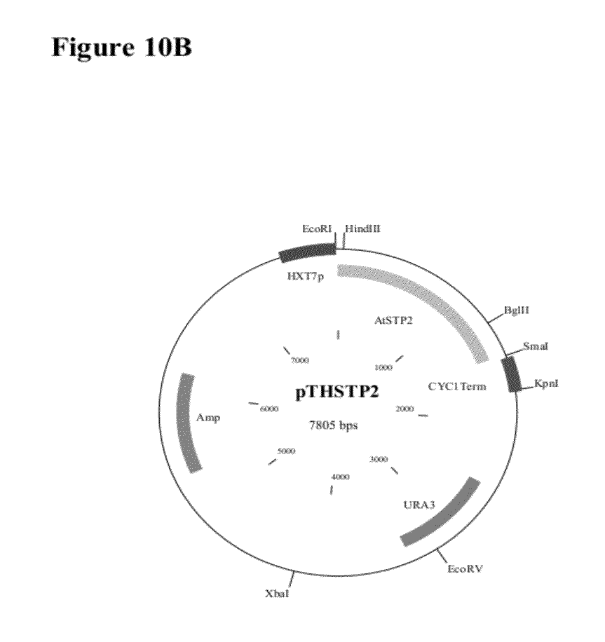

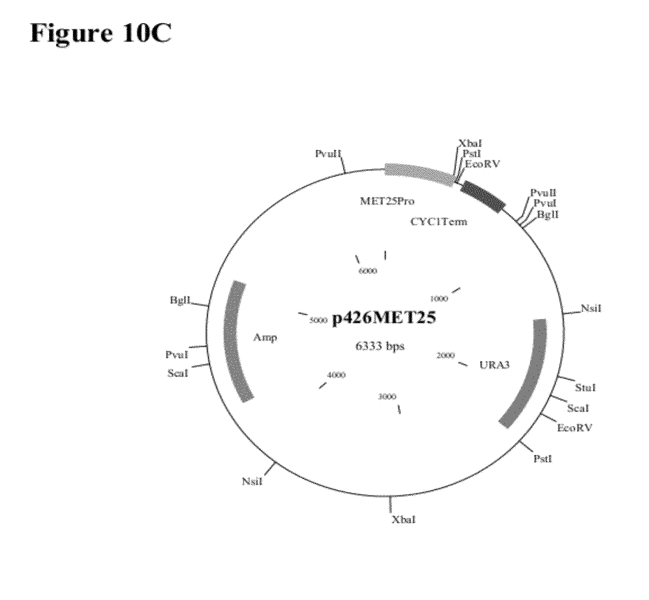

FIG. 10. Vectors used and their structure.

The open reading frame (ORF) of the arabinose transporter Stp2 according to the invention was amplified and cloned behind the shortened strong HXT7 promoter of the plasmid p426HXT7-6HIS (A). With this, the plasmid pTHStp2 (B) was produced, which has a uracil marker. Another possible expression plasmid is p426Met25 (C).

### EXAMPLES

### Methods

### 1. Strains and Media

Bacteria

E. coli SURE (Stratagene)

Full medium LB 1% trypton, 0.5% yeast extract, 0.5% NaCl, pH 7.5 (see Sambrook and Russell, 2001)

For selection on a plasmid-coded antibiotic resistance, 40 μg/ml ampicillin was added to the medium after autoclaving. Solid culture media additionally contained 1.9% agar. The culture took place at 37° C.

Yeast

### Strain EBY.VW4000

EBY.VW4000 (Genotype: MATa leu2-3,112ura3-52 trp1-289 his3-Δ1 MAL2-8c SUC2 Δhxt1-17Δgal2 stlΔ::loxP agt1Δ::loxP mph2Δ::loxP mph3Δ::loxP) (Wieczorke et al., 1999)

### Strain MKY06

MKY06 (Genotype: MATa leu2-3,112 ura3-52 trp1-289 his3-1 MAL2-8c SUC2 hxt1-17 gal2 stl1::loxP agt1::loxP mph2::loxP mph3::loxP PromTAL1::loxP-Prom-vkHXT7, description: EBY.VW4000 PromTAL1::loxP-Prom-vkHXT7)

### Strain MKY06-3P

MKY06-3P (Genotype: MATa leu2-3,112 ura3-52 trp-289 his3-1 MAL2-8c SUC2 hxt1-17 gal2 stl1::loxP agt1::loxP mph2::loxP mph3::loxP PromTAL1::loxP-Prom-vkHXT7, description: EBY.VW4000 PromTAL1::loxP-Prom-vkHXT7); contains the plasmids p423H7-synthIso, p424H7-synthKin and p425H7-synthEpi.

Synthetic Complete Selective Medium SC

0.67% yeast nitrogen base w/o amino acids and ammonium sulphate, 0.5% ammonium sulphate, 20 mM potassium dihydrogenphosphate, pH 6.3, amino acid/nucleobase solution without the corresponding amino acids for the auxotrophy markers of the plasmids used, carbon source in the respectively indicated concentration

Concentration of the amino acids and nucleobases in the synthetic complete medium (Zimmermann, 1975): adenine (0.08 mM), arginine (0.22 mM), histidine (0.25 mM), isoleucine (0.44 mM), leucine (0.44 mM), lysin (0.35 mM), methionine (0.26 mM), phenylalanine (0.29 mM), threonine (0.48 mM), tryptophan (0.19 mM), tyrosin (0.34 mM), uracil (0.44 mM) and valine (0.49 mM). As carbon sources, L-arabinose, D-glucose, D-galactose, D-mannose and maltose were used.

Solid full and selective media contained in addition 1.9% agar. The culture of the yeast cells took place at 30° C.

### 2. Plasmids

| Plasmid | Source/Reference | Description |
|---|---|---|
| p423H7-synthIso | Wiedemann and | Codon-optimised |
| | Boles, 2008. | B. licheniformis araA. in |
| | | p423HXT7- |
| | | 6HIS |
| p424H7-synthKin | Wiedemann and | Codon-optimised E. coli araB |
| | Boles, 2008. | in |
| | | p424HXT7-6HIS, mutation |
| | | in araB |
| p425H7-synthEpi | Wiedemann and | Codon-optimised E. coli araD |
| | Boles, 2008. | in |
| | | p425HXT7-6HIS |
| p426HXT7-6HIS | Becker and Boles, | 2μ plasmid for over-6HIS |
| | 2003 | expression of genes and for |
| | | production of fusion proteins |
| | | with 6xHis-Epitope; URA3 |
| | | marker gene, shortened HXT7 |
| | | promoter and CYC1 |
| | | terminator |
| pHL125^{re} | Liang and Gaber, 1996 | 2μ plasmid with the GAL2 |
| | | gene expressed behind the |
| | | ADH1 promoter, URA3 |
| | | marker gene, reisolated from |
| | | JBY25-4M |
| p426H7-araT | WO 2008/080505 | 2μ plasmid with P. stipitis |
| | | AraT behind the shortened |
| | | HXT7 promoter, URA3 |
| | | marker gene |
| pTHStp2 | Hamacher et al., 2002 | 2μ plasmid expressed with the |
| | | Arabidopis thaliana STP2 |
| | | behind the shortened HXT7 |
| | | promoter, URA3 marker gene |

### 3. Transformation

Transformation of S. Cerevisiae

The transformation of S. cerevisiae was carried out by the lithium-acetate method (Gietz and Woods, 2002).

Transformation of E. Coli

The transformation of the E. coli cells took place by the electroporation method (Dower et al., 1988; Sambrook and Russell, 2001) by means of an Easyject prima apparatus from EQUIBO.

### 4. Preparation of DNA

Isolation of Plasmid-DNA from S. Cerevisiae

The cells of a stationary yeast culture (5 ml) were harvested, washed and re-suspended in 100 μl buffer 1 (taken from the “Plasmid Mini Kit”). After the addidion of 200 μl buffer 2 and 2/3 volume glass beads (diameter=0.45 mm), the cells were solubilised for 8 min on a Vibrax (Janke and Kunkel, Vibrax-VXR) at 4° C. The supernatant was mixed with 150 μl buffer 3 and incubated for 10 min on ice. After centrifuging for 15 minutes at 10000 R/min, the supernatant was used and the plasmid-DNA was precipitated with 400 μl isopropanol (−20° C., 10 min). The DNA, which was pelleted through centrifuging (30 min, 13000 rpm) was washed with 70% cold ethanol and held in 20 μl water. The DNA was then used for a transformation in E. coli or a DNA amplification by means of PCR.

Isolation of Plasmid-DNA from E. Coli

The isolation of plasmid-DNA from E. coli took place with the “Plasmid Mini Kit” of the company Qiagen, according to the manufacturer's information.

Determining the DNA Concentration

The DNA concentration is measured by spectral photometry in a wavelength range of 240-300 nm. If the purity of the DNA, determined by the quotient E260nm/E280 nm is 1.8, then the extinction E260 nm=1.0 corresponds to a DNA concentration of 50 μg dsDNA/ml (Sambrook and Russell, 2001).

### 5. DNA Amplification by Means of PCR

Use of the Expand™ High Fidelity System

The polymerase chain reaction (PCR) took place with the “Expand™ High Fidelity PCR System” of the company Roche, according to the manufacturer's information. 0.2 mM dNTP-mix, 1× buffer 2 (contains 1.5 mM MgC12), 1 U polymerase and 100 pmol each of the corresponding oligonucleotide primers were added together to the plasmid- or genomic DNA to be amplified. The PCR reaction was carried out in a thermocycler (Techne) or mastercycler (Eppendorf).

For the amplification of the DNA, the following temperature cycles were selected.

1. 1× 95° C., 4 min denaturing of the DNA
2. 18-30× 95° C., 30 sec denaturing of the DNA
55° C., 30 sec binding of the primers to the DNA (annealing)
72° C., 2 min DNA synthesis (elongation)
3. 1× 72° C., 4 min synthesis (elongation)

The number of synthesis steps, the annealing temperature and the elongation time were adapted to the specific melting temperatures of the oligonucleotides which were used or to the size of the product which was to be expected. The PCR products were checked by a subsequent agarose gel electrophoresis and then purified.

DNA Purification of PCR Products

The purification of the PCR products took place with the “QIAquick PCR Purification Kit” of the company Qiagen, according to the manufacturer's information.

Gel Electrophoretic Separation of DNA Fragments

The separation of DNA fragments with a size of 0.15-20 kb took place in 1-4% agarose gels. 1xTAE buffer (40 mM Tris, 40 mM acetic acid, 2 mM EDTA) was used as gel- and running buffer (Sambrook and Russell, 2001). Serving as marker was either a lambda phage DNA cut with the restriction endonucleases EcoRI and HindIII, or the 2-log DNA ladder (NEB). Before application, the DNA samples were mixed with 1/10 volume blue marker (1xTAE buffer, 10% glycerine, 0.004% bromophenol blue). After the separation, the gels were incubated in an ethidium bromide bath and the DNA fragments were made visible by irradiation with UV light (254 nm).

Isolation of DNA Fragments from Agarose Gels

The desired DNA fragment was cut out from the TAE agarose gel under longwave UV light (366 nm) and isolated with the “QIAex II Gel Extraction Kit” or the “QIA-quick Gel Extraction Kit” of the company Qiagen, according to the manufacturer's information.

### 6. Enzymatic Modification of DNA

DNA Restriction

Sequence-specific splittings of the DNA with restriction endonucleases were carried out under the incubation conditions recommended by the manufacturer for 2-3 hours with 2-5 U enzyme per μg DNA.

### 7. HPLC Analyses

The samples taken in the tests were centrifuged for 10 min at 3000 R/min, in order to pellet the yeast cells. The supernatant was removed and immediately frozen at −20° C. For the protein precipitation, subsequently 50% sulphosalicylic acid was added, mixed, and centrifuged off for 30 min at 13000 R/min and 4° C. The supernatant was removed, a 1/10 dilution with water was produced therefrom and used for the HPLC analyses. Serving as standards for the measurements were samples with D-glucose, L-arabinose, glycerol, acetate and ethanol, which were used in concentrations of 0.01% w/w, 0.05% w/w, 0.1% w/w, 0.5% w/w and 1.0% w/w. The sugar concentrations were measured by means of BioLC (Dionex). The autosampler “AS50”, the column oven “TCC-100”, the gradient pump “GS50” (all Dionex) and the RI detector “RI-101” (Shodex) were used in the measurement. As a column, the VA 300/7.7 nucleogel sugar 810H (Machery-Nagel) was used with 20% sulphuric acid as eluent (0.6 ml/min). For the evaluation of the analysis data, the Chromeleon™ program (Version 6.50, Dionex) was used.

### 8. Sugar Uptake Analyses

The initial rates of sugar uptake were measured as follows: a 50-0 aliquot of a sugar solution containing (1-³H)-labeled L-arabinose, (U-¹⁴C) labeled O-glucose (American Radiolabeled Chemicals Inc.) or (1-¹⁴C) labeled D-galactose (Amersham) was incubated at 30° C. and was mixed with 100 μl of yeast suspensions having the same temperature, resulting in final sugar concentrations of 10 mM L-arabinose, D-glucose and D-galactose. For determination of L-arabinose uptake kinetics 0.1, 1, 5, 10, and 50 mM L-arabinose were used. After different time intervals 10 ml of ice-cold 100 mM potassium phosphate buffer at pH 6.5 with 500 mM. D-glucose was added, and the suspension was immediately filtrated using Durapore® membrane filters (0.22 μm pore size, Millipore). The filter was washed two times with 10 ml of cold potassium phosphate buffer with 500 mM D-glucose. Filters were transferred to 5 ml scintillation vials containing 4.5 ml. Rotiszint® eco plus (Roth) and the radioactivity measured in a scintillation counter. Uptake of radioactivity was nearly linear in time intervals up to 2 minutes. The results shown are average values for two to three independent experiments. Dry weight was determined by filtering 10 ml of the culture through a pre-weighted nitrocellulose filter (0.45 μm pore size; Roth, Germany). The filters were washed with demineralized water, dried in a microwave oven for 20 minutes at 140 W, and weighted again. K_{M} and vₘₐₓ values were calculated using the program GraphPad Prism 5.0.

### Example 1

### Structure of a Test System for the Examination of Arabinose Transporters

### A) Construction of the MKY06

In the yeast strain EBY.VW4000 all the genes of the hexose transporter family and in addition three genes of the maltose transporter family were deleted. This strain grew on maltose medium unchanged, but was no longer able to grow on glucose, fructose and mannose and only very weakly on galactose (Wieczorke et al., 1999). As all hexose transporters are deleted, it can be assumed that the strain also can no longer receive any pentoses and is therefore suitable for pentose transport investigations.

In preceding tests (see Becker and Boles, 2003), it had been found that in addition to a functional L-arabinose metabolic pathway, also an increased activity of transaldolase was necessary for the use of L-arabinose. For this reason, by exchange of the endogenous promoter of TAL1 in EBY.VW4000 for the shortened HXT7 promoter TAL1 was over-expressed. This strain was named MKY06 and is provided, with the plasmids for the L-arabinose metabolism and a transporter which can transport L-arabinose, to grow on this carbon source.

### B) Introduction of the L-Arabinose Metabolic Pathway

The strain MKY06 was transformed with the plasmids p423H7-synthIso, p424H7-synthKin and p425H7-synthEpi, so that it obtains the capability of L-arabinose use. The transformation with the three plasmids took place simultaneously. The transformants were plated on SC medium with 2% maltose. In a further transformation, as positive control in addition the transporter Gal2, known as L-arabinose transporter, was transformed in and as negative control the empty plasmid p426HXT7-6HIS and plated again on medium plates containing maltose. The positive control, which contains an L-arabinose transporter and the three plasmids for the L-arabinose use and over-expresses transaldolase, should be able to grow on L-arabinose. The negative control should show no growth owing to the absent transporter. This was investigated.

### C) Checking the Test System

In order to be able to use the constructed test system further, firstly the positive and negative controls had to be investigated with regard to their growth. Several colonies of the transformants obtained on the SC plates with 2% maltose were removed with a sterile inoculating loop and smeared on SC plates with 2% L-arabinose and incubated at 30° C. for ten days. After this time, the positive controls showed a distinct growth and the negative control, as expected, showed no growth.

This test system was therefore functional and was able to be used for the investigation of L-arabinose transporters. The positive and negative control mentioned here always served as a comparison in this.

### Example 2

### Growth Behaviour of pTHStp2

The test system was now used in order to test pTHStp2 for growth on medium with different sugar sources.

### A) Carrying Out the Screen

The plasmid pTHStp2 which was constructed and described by Hamacher et al. 2002 was transformed into the strain MKY06-3P and smeared on SC medium with 2% maltose. The colonies obtained after three days at 30° C. were replica-plated on SC medium plates with different carbon sources, in order to test the substrate spectrum.

Growth was only possible when the AtStp2 transporter was able to transport the particular carbon source.

The results are shown in FIG. 4. The strain. MKY06-3P with the pTHStp2 plasmid grows only on plates containing galactose or arabinose and less on mannose. There was no growth on media containing glucose.

In order to be able to rule out genomic mutations which could be responsible for the growth, the colonies which were found were smeared on complete medium with 5-FOA. Thereby, selection was carried out on a loss of the plasmid of the gene bank. With renewed smearing on L-arabinose medium, these colonies of the 5-FOA plate were no longer able to grow hereon. Therefore, a genomic mutation was able to be ruled out as the cause of the growth.

### Example 3

### Characterization of the Arabinose Transporter (AtStp2)

### A) Sequencing

The open reading frame of the AtStp2 in the plasmid pTHStp2 was sequenced with overlapping regions. The sequencing shows that there were no mutations in the open reading frame.

### B) Examples for Vectors for AtStp2

The open reading frame (ORF) of AtStp2 was amplified and cloned behind the shortened strong HXT7 promoter or the plasmid p426HXT7-6HIS. With this, the plasmid pTHStp2 was produced, which has a uracil marker.

Another possible expression plasmid is p426Met25. For vector maps, see FIGS. 10A to 10C.

Further possible expression vectors are pYES260, pYES263, pVTU260, pVTU263, pVTL260 and pVTL263. Further information on these vectors is to be found at http://web.uni-frankfurt.de/fb15/mikro/euroscarf/data/km_expr.html.

### C) Growth Behaviour and Sugar Consumption

The growth of the strain MKY06-3P+pTHStp2 was investigated under aerobic conditions as a function of the L-arabinose concentration and on glucose in the medium. As controls, strains derived from MKY06-3P were used, which additionally also contained the plasmid pHL125^{re}, p426HXT7-6HIS or p426-opt-AraT-S. p426-opt-AraT-S was derived from plasmid p426H7-araT which contains araT, the arabinose transporter of Pichia stipitis as published in WO 2008/080505, by replacing the coding sequence of AraT from Pichia stipitis with a codon-optimised sequence of AraT adapted to the glycolytic codon usage of S. cerevisiae (Wiedemann and Boles 2008).

The strains with the plasmids coding for transporters were adducted in SC medium with 2% L-arabinose and inoculated with an OD₆₀₀ₙₘ=1 in 50 ml SC medium with 0.5%, or 2% L-arabinose, or 2% glucose. The strain containing the empty vector was adducted in SC medium with 1% maltose. The incubation took place in 300 ml shaking flasks under aerobic conditions at 30° C. Samples were taken several times in the day to determine the optical density and the carbon source concentration.

The results are shown in FIGS. 5 to 8. The strain MKY06-3P+pTHStp2 reached a higher optical density on arabinose 2% and 0.5% than on glucose 2% (FIG. 5C). The other strains containing the pHL125^{re} or p426-opt-Ara grew more quickly on glucose media than on arabinose media (FIGS. 5A & 5B).

With an L-arabinose concentration of 2% the strains containing transporters grew more or less similar in the first hours, however, pHL125^{re} reaches a higher optical density at the end of growth with this concentration.

With 0.5% L-arabinose a distinct advantage of pTHStp2 and p426-opt-AraT-S is shown compared with pHL125^{re} (FIGS. 5B & 5C).

These findings were confirmed by the analysis of the sugar consumption (FIGS. 6 to 8). All strains containing arabinose transporters fermented arabinose but only MKY06-3P+pTHStp2 and MKY06-3P+p426-opt-AraT-S consumed arabinose at a concentration of 0.5% arabinose showing that these transporters have a higher affinity to arabinose than Gal2.

While MKY06-3P+pHL125^{re} as well as MKY06-3P+p426-opt-AraT-S fermented glucose, MKY06-3P+pTHStp2 consumed no glucose indicating that this transporter is a specific arabinose transporter which transports arabinose but no glucose.

It is therefore shown that the L-arabinose uptake system according to the invention makes it possible for the recombinant S. cerevisiae cells to use L-arabinose substantially more efficiently.

### D) Sugar Uptake

Furthermore, the initial rates of sugar uptake in strain MKY06 (without the L-arabinose utilization pathway) overexpressing Stp2 or Gal2 were measured with radioactively labeled sugars (see FIG. 9). Cells were pre-grown in SC medium with D-galactose (cells expressing transporters) or maltose (empty vector). L-arabinose, D-galaetose and D-glucose uptake rates were measured during 1- or 2-minute time intervals, respectively, at 10 mM final sugar concentrations. Uptake of D-galactose was mediated by both transporters. Whereas a high rate of D-glucose uptake could be measured with cells expressing Gal2 (4.7 nmol/min/mg dry mass (DM)), D-glucose uptake mediated by Stp2 was hardly to detect (<0.01 nmol/min/mg DM).

For L-arabinose, uptake kinetics were determined by measuring L-arabinose uptake at various concentrations between 0.1 and 50 mM during 2-minute time intervals. While Gal2 turned out to transport L-arabinose with low affinity and high capacity, Stp2 mediated uptake of L-arabinose with low capacity but high affinity (Table 1). In both cases, addition of 10 mM D-galactose or D-glucose nearly completely inhibited L-arabinose uptake.

TABLE 1
K_{M} and vₘₐₓ values for D-galactose/L-arabinose
transporters and L-arabinose as the substrate

| Transporter | Gal2 | Stp2 |
|---|---|---|
| K_{M} (mM) | 57 +/− 11 | 4.5 +/− 2.2 |
| vₘₐₓ (nmol/min/mg DM) | 2.2 +/− 0.26 | 0.6 +/− 0.08 |

K_{M}: Michaelis constant;
vₘₐₓ: maximal enzyme reaction velocity

The determination of L-arabinose uptake kinetics revealed that, whereas Gal2 turned out to have a relatively low affinity but high capacity for L-arabinose, AtStp2 exhibited higher affinities but lower capacities. These characteristics were clearly reflected in the growth properties of the strains expressing the individual transporters on different L-arabinose concentrations. Gal2 supported growth on L-arabinose only at high concentrations, reflecting its low affinity; Stp2 did so especially at low concentrations due to its higher affinities.

Until now, Gal2 was the only transporter used to increase L-arabinose uptake in recombinant S. cerevisiae fermenting L-arabinose. Either targeted overexpression of ScGal2 improved L-arabinose utilization or expression of GAL2 was increased by evolutionary engineering of a yeast strain for improved fermentation of L-arabinose. Also in this work, we could show that at high L-arabinose concentrations Gal2 efficiently catalyzes L-arabinose uptake. Nevertheless, in many sources of plant biomass L-arabinose is present in only minor amounts. Interestingly, the newly discovered L-arabinose transporter Stp2 supports efficient uptake of L-arabinose especially at low L-arabinose concentrations, in contrast to Gal2. Thus, the use of Stp2 can improve the fermentation of the low L-arabinose concentrations in typical lignocellulosic hydrolysates after the D-glucose has been consumed.

### REFERENCES

Becker, J. and Boles, E. (2003) A modified Saccharomyces cerevisiae strain that consumes L-Arabinose and produces ethanol. Appl Environ Microbiol 69(7),4144-50.
Boles, E. (2002) in “Transmembrane Transporters”, Editor Michael W. Quick, WILEY-LISS, 19-36.
Dien, B. S., Kurtzman, C. P., Saha, B. C. and Bothast, R. J. (1996) Screening for L-arabinose fermenting yeasts, Appl Biochem Biotechnol 57-58, 233-42.
Dower, W. J., Miller, J. F. and Ragsdale, C. W. (1988) High efficiency transformation of E. coli by high voltage electroporation. Nucleic Acids Res 16(13), 6127-45.
Gietz, R. D. and Woods, R. A. (2002) Transformation of yeast by lithium acetate/single-stranded carrier DNA/polyethylene glycol method. Methods Enzymol 350, 87-96.
Hahn-Hagerdal, B., Wahlbom, C. F., Gardonyi, M., van Zyl, W. H., Cordero Otero, R. R. and Jonsson, L. J. (2001) Metabolic engineering of Saccharomyces cerevisiae for xylose utilization. Adv Biochern Eng Biotechnol 73, 53-84.
Hamacher T., Becker J., Gárdonyi M., Hahn-Hagerdal B., Boles E. (2002) Characterization of the xylose-transporting properties of yeast hexose transporters and their influence on xylose utilization. Microbiology 148 (9):2783-8
Jeppson M, Bengtsson O, Franke K, Lee H, Hahn-Hagerdal B, Gorwa-Grauslund M F. (2006) The expression of a Pichia stipitis xylose reductase mutant with higher K(M) for NADPH increases ethanol production from xylose in recombinant Saccharomyces cerevisiae. Biotechnol Bioeng. 93 (4):665-73.
Jin Y S, Jeffries T W. (2004) Stoichiometric network constraints on xylose metabolism by recombinant Saccharomyces cerevisiae. Metab Eng. 6(3):229-38.
Katahira S, Mizuike A, Fukuda H, Kondo A. (2006) Ethanol fermentation from lignocellulosic hydrolysate by a recombinant xylose- and cellooligosaccharide-assimiliating yeast strain. Appl Microbiol Biotechnol. 72(6):1136-43.
Kotter, P. and Ciriacy, M. (1993) Xylose fermentation by Sacharomyces cerevisiae. Appl Microbiol Biotechnol 38, 776-783.
Kou, S. C., Christensen, M. S. and Cirillo, V. P. (1970) Galactose transport in Saccharomyces cerevisiae. II. Characteristics of galactose uptake and exchange in galactokinaseless cells. J Bacteriol 103(3), 671-8.
Kuyper. M., Toirkens, M. J., Diderich, J. A., Winkler. A. A., van Dijken, J. P. and Pronk, J. T. (2005b) Evolutionary engineering of mixed-sugar utilization by a xylose-fermenting Saccharomyces cerevisiae strain. FEMS Yeast Res 5(10), 925-34.
Kuyper, M., Winkler, A. A., van Dijken, J. P. and Pronk, J. T. (2004) Minimal metabolic engineering of Saccharomyces cerevisiae for efficient anaerobic xylose fermentation: a proof of principle. FEMS Yeast Res 4(6), 655-64.
Liang, H. and Gaber, R. F. (1996) A novel signal transduction pathway in Saccharomyces cerevisiae defined by Snf3-regulated expression of HXT6. Mol Biol Cell 7(12):1953-66.
Lucas, C. and Uden, N. v. (1986) Transport of hemicellulose monomers in the xylose-fermenting yeast Candida shehatae. Appl Microbiol Biotechnol 23, 491-495.
Sambrook J. and Russell D. 2001) Molecular cloning. A laboratory manual. New York: Cold Spring Harbor
Pitkanen J P, Rintala E, Aristidou A, Ruohonen L, Penttila M. (2005) Xylose chemostat isolates of Saccharomyces cerevisiae show altered metabolite and enzyme levels compared with xylose, glucose, and ethanol metabolism of the original strain. Appl Microbiol Biotechnol. 67(6):827-37.
Richard, P., Putkonen, M., Vaananen, R., Londesborough, J. and Penttila, M. (2002) The missing link in the fungal L-arabinose catabolic pathway, identification of the L-xylulose reductase gene. Biochemistry 41(20), 6432-7.
Truernit E., Stadler R., Baier K., Sauer N. (1999) A male gametophyte-specific monosaccharide transporter in Arabidopsis. Plant J. 17 (2):191-201.
Verho R, Penttilä M. Richard P. Cloning of Two Genes (LAT1,2) Encoding Specific L: -Arabinose Transporters of the L: -Arabinose Fermenting Yeast Ambrosiozyma monospora. Appl Biochem Biotechnol. 2011 Jan. 21. [Epub ahead of print] DOI 10.1007/s12010-011-9161-y
Wieczorke, R., Krampe, S., Weierstall, T., Freidel, K., Hollenberg, C. P. and Boles, E. (1999) Concurrent knock-out of at least 20 transporter genes is required to block uptake of hexoses in Saccharaomyces cerevisiae. FEBS Lett 464(3), 123-8.
Wiedemann B., Boles E. (2008) Codon-optimized bacterial genes improve L-Arabinose fermentation in recombinant Saccharomyces cerevisiae. Appl Environ Microbiol. 74 (7):2043-50
Zaldivar, J., Nielsen, J. and Olsson, L. (2001) Fuel ethanol production from lignocellulose: a challenge for metabolic engineering and process integration. Appl Microbiol Biotechnol 56(1-2), 17-34.
Zimmermann, F. K. (1975) Procedures used in the induction of mitotic recombination and mutation in the yeast Saccharomyces cerevisiae. Mutat Res 31(2), 71-86.

We claim:

1. A method for conferring upon a cell the ability to take up L-arabinose, wherein said method comprises transforming the cell with a nucleic acid molecule comprising a nucleic acid sequence, which codes for a plant pentose transporter, wherein the transformation enables the cell to take up L-arabinose, wherein the cell is a yeast cell, and wherein the plant pentose transporter originates from Arabidopsis and comprises an amino acid sequence-that is at least 70% identical to the amino acid sequence of SEQ ID NO: 1 and has an in vitro and/or an in vivo pentose transport function, or a functional equivalent of SEQ ID NO: 1 having an in vitro and/or an in vivo pentose transport function.

2. The method according to claim 1, wherein the plant pentose transporter in vitro and/or in vivo transports L-arabinose and does not transport D-glucose.

3. The method according to claim 1, wherein the plant pentose transporter has an amino acid sequence of SEQ ID NO: 1, or is a functional equivalent thereof having an in vitro and/or an in vivo pentose transport function.

4. The method according to claim 1, wherein the nucleic acid sequence coding for a plant pentose transporter comprises a nucleic acid sequence that is at least 70% identical to the nucleic acid sequence of SEQ ID NO: 2.

5. The method according to claim 1, wherein the nucleic acid molecule is a nucleic acid expression construct, which comprises regulatory sequences being operatively linked with the nucleic acid sequence coding for the plant pentose transporter, and/or wherein the nucleic acid expression construct further comprises 5′ and/or 3′ recognition sequences and/or selection markers.

6. The method according to claim 1, wherein the yeast cell is a member of a genus selected from the group consisting of Saccharomyces, Kluyveromnyces, Candida, Pichia, Schizosaccharomyces, Hansenula, Kloeckera, Schwanniomyces, Arxula and Yarrowia.

7. The method, according to claim 6, wherein the yeast cell is a member of a species selected from the group consisting of S. cerevisiae, S. bulderi, S. barnetti, S. exiguus, S. uvarum, S. diastaticus, K. lactis, K. marxianus, K. fragilis, H. polymorpha, P. pastoris and Y. lipolytica.

8. The method according to claim 1, wherein the cell further contains nucleic acid sequences coding for proteins of an arabinose metabolic pathway.

9. The method according to claim 1, wherein said method is used for
the conversion and metabolization of biomaterial containing pentose(s),
the conversion and metabolization of biomaterial containing hexoses and pentoses,
the production of bio-based chemicals or biofuels, and/or
the production of bioalcohols.

10. The method, according to claim 9, wherein the biomaterial contains the pentose L-arabinose, and/or the hexose D-glucose, and/or D-xylose.

11. The method, according to claim 9, used to produce bioethanol and/or biobutanol.

12. A yeast cell, which is transformed with a nucleic acid expression construct comprising:
(a) a nucleic acid sequence coding for a plant pentose transporter, wherein the plant pentose transporter originates from Arabidopsis and comprises an amino acid sequence that is at least 70% identical to the amino acid sequence of SEQ ID NO: 1 and has an in vitro and/or an in vivo pentose transport function, or a functional equivalent of SEQ ID NO: 1 having an in vitro and/or an in vivo pentose transport function, and
(b) regulatory elements operatively linked with the nucleic acid sequence, allowing for the expression of the plant pentose transporter in the yeast cell, wherein the expression of the nucleic acid expression construct imparts to the yeast cell the capability to take up L-arabinose.

13. The yeast cell according to claim 12, wherein the expression of the nucleic acid expression construct imparts to the yeast cell the capability to take up L-arabinose but not D-glucose.

14. The yeast cell according to claim 13, wherein the yeast cell is transformed with a nucleic acid expression construct, which comprises regulatory sequences being operatively linked with the nucleic acid sequence coding for the plant pentose transporter, and/or wherein the nucleic acid expression construct further comprises 5′ and/or 3′ recognition sequences and/or selection markers.

15. The yeast cell according to claim 12, furthermore expressing one or more enzymes, which impart to the cell the capability to produce one or more further metabolization products.

16. A method for
the conversion and metabolization of biomaterial containing pentose(s),
the conversion and metabolization of biomaterial containing hexoses and pentoses,
the production of bio-based chemicals or biofuels, and/or
the production of bioalcohols wherein said method comprises the use of a yeast cell according to claim 12.

17. The method, according to claim 16, wherein the biomaterial contains the pentose arabinose, and/or the hexose D-glucose, and/or D-xylose.

18. The method, according to claim 16, used to produce bioethanol and/or biobutanol.

19. A method for the production of a bioalcohol comprising the steps of:
(a) contacting a medium containing a pentose source with a cell according to claim 12, which converts pentose to a bioalcohol, and
(b) optionally obtaining the bioalcohol.

20. The method, according to claim 19, wherein the pentose is L-arabinose.

21. The method according to claim 19, wherein the medium contains a further carbon source.

22. A method for the production of a metabolization product comprising the steps of:
(a) contacting a medium containing a pentose source with a cell according to claim 12, which converts pentose to produce the metabolization product, and
(b) optionally obtaining the metabolization product.

23. The method according to claim 22, wherein the medium contains a further carbon source.

24. The method, according to claim 22, wherein the metabolization product is selected from the group consisting of lactic acid, acetic acid, succinic acid, malic acid, 1-butanol, isobutanol, 2-butanol, amino acids, 1,3-propanediol, ethylene, glycerol, β-lactam antibiotics, cephalosporins, alkanes, terpenes, isoprenoids and the precursor molecule amorphadiene of the antimalarial drug artemisinin, and wherein the pentose is L-arabinose.


SEQUENCE LISTING (2 sequences)

SEQ ID NO: 1 - protein, 498 aa, Arabidopsis thaliana
MAVGSMNVEEGTKAFPAKLTGQVFLCCVIAAVGGLMFGYDIGISGGVTSMDTFLLDFFPHVYEKKHRVHENNYCKFDDQLLQLFTSSLYLAGIFASFISSYVSRAFGRKPTIMLASIFFLVGAILNLSAQELGMLIGGRILLGFGIGFGNQTVPLFISEIAPARYRGGLNVMFQFLITIGILAASYVNYLTSTLKNGWRYSLGGAAVPALILLIGSFFIHETPASLIERGKDEKGKQVLRKIRGIEDIELEFNEIKYATEVATKVKSPFKELFTKSENRPPLVCGTLLQFFQQFTGINVVMFYAPVLFQTMGSGDNASLISTVVTNGVNAIATVISLLVVDFAGRRCLLMEGALQMTATQMTIGGILLAHLKLVGPITGHAVPLIVLILICVYVSGFAWSWGPLGWLVPSEIYPLEVRNAGYFCAVAMNMVCTFIIGQFFLSALCRFRSLLFFFFGIMNIIMGLFVVFFLPETKGVPIEEMAEKRWKTHPRWKKYFKD

SEQ ID NO: 2 - DNA, 1497 bp, Arabidopsis thaliana
atggctgttggttcgatgaatgtcgaagaaggcactaaagcttttcctgcgaagctgacaggtcaagtgtttctttgctgtgtcattgctgctgttggtggtctcatgtttggttacgacatcggaatctcaggaggtgtgacgagtatggacactttcttgttagattttttcccacacgtgtacgagaagaaacacagagtacacgaaaacaactactgcaaattcgatgaccagctcttgcagttgttcacttcctcactctacttggctggaatcttcgctagctttatttcttcctatgtttctagggcttttggaagaaaacccacaatcatgctcgcctccatcttcttcctcgtcggtgctatcctcaacttatctgcccaagaactcggcatgttgatcggtggtcgtattcttctcggctttggtatcggtttcggtaaccagaccgttccattgttcatctcagagattgctccggcgagatacagaggaggactaaacgtcatgttccagtttctcatcaccattggaatcttagcagcaagttatgtgaactacttaacttccacgttgaaaaacggctggagatattctctcggtggtgctgctgtccccgctttaatcctcttgataggatccttctttatccacgagactccggctagtctcatcgagcgcggtaaagacgaaaaggggaagcaagtcctgaggaagatcagaggcattgaagatattgagctcgagttcaatgagatcaaatacgcaacagaggtagcgacaaaggtgaaaagcccattcaaagaactcttcaccaagagtgagaacagaccaccattggtgtgtggaacgttacttcagttctttcaacagttcaccggtatcaacgtggttatgttctacgctccagttttgttccagacgatgggtagtggtgacaacgcttctctgatctctactgttgtcaccaatggtgtgaacgcaatcgctacagttatctctcttttagtggttgatttcgccggtagaaggtgtcttctgatggaaggagctctccagatgaccgctacacagatgacaattggaggcattctcttagctcacttgaagctagttggtcctattactggccatgccgtgccgctgattgtattgatcctcatctgcgtatacgtgtctggatttgcatggtcttggggtccattgggatggcttgttccgtctgagatctaccctcttgaagtgagaaatgctggttacttctgtgcggtggcgatgaacatggtctgcactttcatcattggacagttcttcttgtctgctctctgcagattcagatcattattgttcttcttcttcggaataatgaacattatcatgggactatttgtcgtgttttttcttcccgaaaccaaaggtgttcctattgaggaaatggccgagaagcgttggaaaacgcacccgcgttggaagaaatatttcaaagactag